(19) United States
(12) Patent Application Publication
(10) Pub. No.: US 2014/0073511 A1
(43) Pub. Date: Mar. 13, 2014

(54) System and Method for Operation of Isfet Arrays Using pH Inert Reference Sensors

(71) Applicant: 454 LIFE SCIENCES CORPORATION, Branford, CT (US)

(72) Inventors: Chiu Tai Andrew Wong, Orange, CT (US); Gianni Calogero Ferreri, Northford, CT (US); Sagnik Basuray, Columbia, MO (US); Arthika Bappal, Bridgeport, CT (US); Suresh Gopalkrishna Shenoy, Branford, CT (US); Xavier Victor Gomes, Wallingford, CT (US)

(21) Appl. No.: 13/970,167

(22) Filed: Aug. 19, 2013

Related U.S. Application Data
(60) Provisional application No. 61/698,018, filed on Sep. 7, 2012.

(51) Int. Cl. C12Q 1/68; H01L 27/105

(52) U.S. Cl. CPC C12Q 1/6874; H01L 27/105
USPC 506/2; 506/16; 257/253

(57) ABSTRACT

An embodiment of a method for sequencing a species of nucleic acid template using pH inert reference sensors is described that comprises the steps of: introducing a nucleotide species to an array of wells where a plurality of the wells comprise a species of nucleic acid template and a plurality of the wells comprise a plurality of functional groups with a high pH buffering characteristic, and in at least a first well a polymerase species incorporates the nucleotide species into a plurality of strands complementary to the species of nucleic acid template disposed in the first well and results in a release of a plurality of hydrogen ions; detecting a signal in the first well that is responsive to the hydrogen ions and one or more noise sources; detecting a signal in a second well comprising the functional groups with the high pH buffering characteristic that is responsive to the one or more noise sources; and subtracting the second well signal from the first well signal to generate a corrected signal associated with the detected hydrogen ions.

### CROSS-REFERENCE TO RELATED APPLICATIONS

This application is related to and claims priority from U.S. Provisional Patent Application Ser. No. 61/698,018, titled “System and Method for Operation of ISFET Arrays Using pH Inert Reference sensors”, filed Sep. 7, 2012, which is hereby incorporated by reference herein in its entirety for all purposes.

### FIELD OF THE INVENTION

The present invention relates to the fields of semiconductor based detection and molecular biology. More specifically, the invention relates to a system and method comprising an ISFET based detection platform comprising wells for sequencing nucleic acid template molecules.

### BACKGROUND OF THE INVENTION

Sequencing-by-synthesis (SBS) generally refers to methods for determining the identity or sequence composition of one or more nucleotides in a nucleic acid sample, wherein the methods comprise the stepwise synthesis of a single strand of polynucleotide molecule complementary to a template nucleic acid molecule whose nucleotide sequence composition is to be determined. For example, SBS techniques typically operate by adding a single nucleic acid (also referred to as a nucleotide) species to a nascent polynucleotide molecule complementary to a nucleic acid species of a template molecule at a corresponding sequence position. The addition of the nucleic acid species to the nascent molecule is generally detected using a variety of methods known in the art that include, but are not limited to what are referred to as pyrosequencing which may include enzymatic detection, semiconductor based sequencing methods employing electronic detection (i.e. pH detection with ISFET or other related technology) detection strategies or fluorescent detection methods that in some embodiments may employ reversible terminators. Typically, the process is iterative until a complete (i.e. all sequence positions are represented) or desired sequence length complementary to the template is synthesized. Some examples of SBS techniques are described in U.S. Pat. Nos. 6,274,320, 7,211,390; 7,244,559; 7,264,929; and 7,335,762 each of which is hereby incorporated by reference herein in its entirety for all purposes.

In some embodiments of SBS, an oligonucleotide primer is designed to anneal to a predetermined, complementary position of the sample template molecule. The primer/template complex is presented with a nucleotide species in the presence of a nucleic acid polymerase enzyme. If the nucleotide species is complementary to the nucleic acid species corresponding to a sequence position on the sample template molecule that is directly adjacent to the 3′ end of the oligonucleotide primer, then the polymerase will extend the primer with the nucleotide species.

As described above, incorporation of the nucleotide species by a polymerase results in a release of Hydrogen (H⁺) that can be detected by elements sensitive to changes in pH, such as semiconductor based Ion Sensitive Field Effect Transistor (hereafter referred to as ISFET) technologies examples of which are described in U.S. Pat. Nos. 7,686,929 and 7,649,358, each of which is hereby incorporated by reference herein in its entirety for all purposes. However, typical ISFET embodiments are also sensitive to other conditions that can create a detectable surface potential change on the ISFET sensing layer that may include some background effects such as temperature related changes and electrical signals present in the environment. Solutions to eliminate signal noise created by these background effects have been developed using what are known are reference sensors, also referred to as reference FET (or REFET). Examples of REFET solutions are described in P. Bergveld et al., “How electrical and chemical requirements for refets coincide”, Sensors and Actuators 18, no. 3-4 (July 1989): 309-327; and A. Errachid, J. Bausells, and N. Jaffrezic-Renault, “A simple REFET for pH detection in differential mode,” Sensors and Actuators B: Chemical 60, no. 1 (Nov. 2, 1999): 43-48, each of which is hereby incorporated by reference herein in its entirety for all purposes.

In embodiments enabled for sequencing nucleic acid molecules in a massively parallel way, arrays of well structures each having an ISFET sensor disposed at the bottom surface have been developed. It is therefore desirable to develop an inexpensive and easily executable approach to creating reference sensors in the array of wells that can be used for subtraction of background noise signals to improve discrimination of signals generated by very small pH changes. Also in the same or alternative embodiments, it is desirable to develop solutions that minimize transmission of H⁺ between wells in the array that is sometimes referred to as “crosstalk” and results in spurious signals from neighboring wells.

A number of references are cited herein, the entire disclosures of which are incorporated herein, in their entirety, by reference for all purposes. Further, none of these references, regardless of how characterized, is admitted as prior art to the invention of the subject matter claimed herein.

### SUMMARY OF THE INVENTION

Embodiments of the invention relate to the determination of the sequence of nucleic acids. More particularly, embodiments of the invention relate to using pH buffering substrates in semiconductor based sequencing systems to create reference sensors and reduce background noise signals.

An embodiment of a method for sequencing a species of nucleic acid template using pH inert reference sensors is described that comprises the steps of: introducing a nucleotide species to an array of wells where a plurality of the wells comprise a species of nucleic acid template and a plurality of the wells comprise a plurality of functional groups with a high pH buffering characteristic, and in at least a first well a polymerase species incorporates the nucleotide species into a plurality of strands complementary to the species of nucleic acid template disposed in the first well and results in a release of a plurality of hydrogen ions; detecting a signal in the first well that is responsive to the hydrogen ions and one or more noise sources; detecting a signal in a second well comprising the functional groups with the high pH buffering characteristic that is responsive to the one or more noise sources; and subtracting the second well signal from the first well signal to generate a corrected signal associated with the detected hydrogen ions.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the figure in which the references element first appears (for example, element 150 appears first in FIG. 1). All of these conventions, however, are intended to be typical or illustrative, rather than limiting.

FIG. 1 is a functional block diagram of one embodiment of a sequencing instrument under computer control and a reaction substrate;

FIG. 2 is a simplified graphical representation of one embodiment of an ISFET well structure and a bead;

FIG. 3 is a simplified graphical representation of one embodiment of signal traces detected by an ISFET sensor over an acquisition time as a function of temperature;

FIGS. 4A and 4B are simplified graphical representations of one embodiment of a plurality of ISFET well structures, pH buffering beads, and signal traces detected by a plurality of ISFET sensors over an acquisition time as a function of pH buffering;

FIGS. 5A and 5B are simplified graphical representations of one embodiment of a comparison of response time signals from a plurality of ISFET sensors as a function of high buffering beads versus low buffering beads;

FIG. 6 is a simplified graphical representation of one embodiment of ISFET signals acquired from Polyethylene glycol (PEG) beads functionalized with carboxylic acid and non-functionalized beads;

FIGS. 7A and 7B are simplified graphical representations of one embodiment of a plurality of ISFET well structures, nucleic acid template beads, pH buffering beads signal, and signal traces demonstrating subtraction of noise signals;

FIG. 8 is a simplified graphical representation of one embodiment of and array of wells comprising pH buffering beads and nucleic acid beads;

FIG. 9 is a simplified graphical representation of two embodiments of a 2 bead layer strategy and one embodiments of a 3 bead layer strategy;

FIGS. 10A, 10B, and 10C are simplified graphical representations of one embodiment of a comparison of signals acquired when using high pH buffering beads, packing beads, and nucleic acid beads in the layering strategies of FIG. 9; and

FIG. 11 is a simplified graphical representation of one embodiment of ISFET signals detected from wells that are adjacent to a well with detectable pH change signals.

### DETAILED DESCRIPTION OF THE INVENTION

As will be described in greater detail below, embodiments of the presently described invention include systems and methods comprising an ISFET based detection platform comprising wells for sequencing nucleic acid template molecules. In the embodiments described in detail below species of template nucleic acid are disposed in an array of wells where a well typically comprises a single species of template nucleic acid. In one described embodiment one or more wells do not have a species of template nucleic acid, instead having a high pH buffering substrate disposed therein which is employed as a reference well in methods that process signals detected from wells that comprise template nucleic acid. Also, in some embodiments high pH buffering substrates are employed to reduce well to well transmission of H⁺ ions that are the byproduct of reactions detected by the ISFET detectors.

### a. GENERAL

Unless otherwise defined, all technical and scientific terms used herein have the same meaning as commonly understood by one of ordinary skill in the art to which this invention belongs. Methods and materials similar or equivalent to those described herein can be used in the practice of the present invention, and exemplified suitable methods and materials are described below. For example, methods may be described which comprise more than two steps. In such methods, not all steps may be required to achieve a defined goal and the invention envisions the use of isolated steps to achieve these discrete goals. The disclosures of all publications, patent applications, patents, and other references are incorporated in toto herein by reference. In addition, the materials, methods, and examples are illustrative only and not intended to be limiting.

The term “flowgram” generally refers to a graphical representation of sequence data generated by SBS methods, particularly pyrophosphate based sequencing methods (also referred to as “pyrosequencing”) and may be referred to more specifically as a “pyrogram”.

The term “read” or “sequence read” as used herein generally refers to the entire sequence data obtained from a single nucleic acid template molecule or a population of a plurality of substantially identical copies of the template nucleic acid molecule.

The terms “run” or “sequencing run” as used herein generally refer to a series of sequencing reactions performed in a sequencing operation of one or more template nucleic acid molecules.

The term “flow” as used herein generally refers to a single introduction of a nucleotide species or reagent into a reaction environment that is typically part of an iterative sequencing by synthesis process comprising a template nucleic acid molecule. For example, a flow may include a solution comprising a nucleotide species and/or one or more other reagents, such as buffers, wash solutions, or enzymes that may be employed in a sequencing process or to reduce carryover or noise effects from previous flows of nucleotide species.

The term “flow order”, “flow pattern”, or “nucleotide dispensation order” as used herein generally refers to a pre-determined series of flows of a nucleotide species into a reaction environment. In some embodiments a flow cycle may include a sequential addition of 4 nucleotide species in the order of T, A, C, G nucleotide species, or other order where one or more of the nucleotide species may be repeated.

The term “flow cycle” as used herein generally refers to an iteration of a flow order where in some embodiments the flow cycle is a repeating cycle having the same flow order from cycle to cycle, although in some embodiments the flow order may vary from cycle to cycle.

The term “read length” as used herein generally refers to an upper limit of the length of a template molecule that may be reliably sequenced. There are numerous factors that contribute to the read length of a system and/or process including, but not limited to the degree of GC content in a template nucleic acid molecule.

The term “signal droop” as used herein generally refers to a decline in detected signal intensity as read length increases.

The term “test fragment” or “TF” as used herein generally refers to a nucleic acid element of known sequence composition that may be employed for quality control, calibration, or other related purposes.

The term “primer” as used herein generally refers to an oligonucleotide that acts as a point of initiation of DNA synthesis under conditions in which synthesis of a primer extension product complementary to a nucleic acid strand is induced in an appropriate buffer at a suitable temperature. A primer is preferably a single stranded oligodeoxyribonucleotide.

A “nascent molecule” generally refers to a DNA strand which is being extended by the template-dependent DNA polymerase by incorporation of nucleotide species which are complementary to the corresponding nucleotide species in the template molecule.

The terms “template nucleic acid”, “template molecule”, “target nucleic acid”, or “target molecule” generally refer to a nucleic acid molecule that is the subject of a sequencing reaction from which sequence data or information is generated.

The term “nucleotide species” as used herein generally refers to the identity of a nucleic acid monomer including purines (Adenine, Guanine) and pyrimidines (Cytosine, Uracil, Thymine) typically incorporated into a nascent nucleic acid molecule. “Natural” nucleotide species include, e.g., adenine, guanine, cytosine, uracil, and thymine. Modified versions of the above natural nucleotide species include, without limitation, alpha-thio-triphosphate derivatives (such as dATP alpha S), hypoxanthine, xanthine, 7-methylguanine, 5,6-dihydrouracil, and 5-methylcytosine.

The term “monomer repeat” or “homopolymers” as used herein generally refers to two or more sequence positions comprising the same nucleotide species (i.e. a repeated nucleotide species).

The term “homogeneous extension” as used herein generally refers to the relationship or phase of an extension reaction where each member of a population of substantially identical template molecules is homogenously performing the same extension step in the reaction.

The term “completion efficiency” as used herein generally refers to the percentage of nascent molecules that are properly extended during a given flow.

The term “incomplete extension rate” as used herein generally refers to the ratio of the number of nascent molecules that fail to be properly extended over the number of all nascent molecules.

The term “genomic library” or “shotgun library” as used herein generally refers to a collection of molecules derived from and/or representing an entire genome (i.e. all regions of a genome) of an organism or individual.

The term “amplicon” as used herein generally refers to selected amplification products, such as those produced from Polymerase Chain Reaction or Ligase Chain Reaction techniques.

The term “variant” or “allele” as used herein generally refers to one of a plurality of species each encoding a similar sequence composition, but with a degree of distinction from each other. The distinction may include any type of variation known to those of ordinary skill in the related art, that include, but are not limited to, polymorphisms such as single nucleotide polymorphisms (SNPs), insertions or deletions (the combination of insertion/deletion events are also referred to as “indels”), differences in the number of repeated sequences (also referred to as tandem repeats), and structural variations.

The term “allele frequency” or “allelic frequency” as used herein generally refers to the proportion of all variants in a population that is comprised of a particular variant.

The term “key sequence” or “key element” as used herein generally refers to a nucleic acid sequence element (typically of about 4 sequence positions, i.e., TGAC or other combination of nucleotide species) associated with a template nucleic acid molecule in a known location (i.e., typically included in a ligated adaptor element) comprising known sequence composition that is employed as a quality control reference for sequence data generated from template molecules. The sequence data passes the quality control if it includes the known sequence composition associated with a Key element in the correct location.

The term “keypass” or “keypass well” as used herein generally refers to the sequencing of a full length nucleic acid test sequence of known sequence composition (i.e., a “test fragment” or “TF” as referred to above) in a reaction well, where the accuracy of the sequence derived from TF sequence and/or Key sequence associated with the TF or in an adaptor associated with a target nucleic acid is compared to the known sequence composition of the TF and/or Key and used to measure of the accuracy of the sequencing and for quality control. In typical embodiments, a proportion of the total number of wells in a sequencing run will be keypass wells which may, in some embodiments, be regionally distributed.

The term “blunt end” as used herein is interpreted consistently with the understanding of one of ordinary skill in the related art, and generally refers to a linear double stranded nucleic acid molecule having an end that terminates with a pair of complementary nucleotide base species, where a pair of blunt ends are typically compatible for ligation to each other.

The term “sticky end” or “overhang” as used herein is interpreted consistently with the understanding of one of ordinary skill in the related art, and generally refers to a linear double stranded nucleic acid molecule having one or more unpaired nucleotide species at the end of one strand of the molecule, where the unpaired nucleotide species may exist on either strand and include a single base position or a plurality of base positions (also sometimes referred to as “cohesive end”).

The term “SPRI” as used herein is interpreted consistently with the understanding of one of ordinary skill in the related art, and generally refers to the patented technology of “Solid Phase Reversible Immobilization” wherein target nucleic acids are selectively precipitated under specific buffer conditions in the presence of beads, where said beads are often carboxylated and paramagnetic. The precipitated target nucleic acids immobilize to said beads and remain bound until removed by an elution buffer according to the operator's needs (DeAngelis, Margaret M. et al: Solid-Phase Reversible Immobilization for the Isolation of PCR Products. Nucleic Acids Res (1995), Vol. 23:22; 4742-4743, which is hereby incorporated by reference herein in its entirety for all purposes).

The term “carboxylated” as used herein is interpreted consistently with the understanding of one of ordinary skill in the related art, and generally refers to the modification of a material, such as a microparticle, by the addition of at least one carboxl group. A carboxyl group is either COOH or COO—.

The term “paramagnetic” as used herein is interpreted consistently with the understanding of one of ordinary skill in the related art, and generally refers to the characteristic of a material wherein said material's magnetism occurs only in the presence of an external, applied magnetic field and does not retain any of the magnetization once the external, applied magnetic field is removed.

The term “bead” or “bead substrate” as used herein generally refers to any type of solid phase particle of any convenient size, of irregular or regular shape and which is fabricated from any number of known materials such as cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like (as described, e.g., in Merrifield, Biochemistry 1964, 3, 1385-1390), polyacrylamides, latex gels, polystyrene, dextran, rubber, silicon, plastics, nitrocellulose, natural sponges, silica gels, control pore glass, metals, cross-linked dextrans (e.g., Sephadex™) agarose gel (Sepharose™), and other solid phase bead supports known to those of skill in the art although it will be appreciated that solid phase substrates may include a degree of porosity enabling penetration of fluids and/or biological molecule into the pores.

The term “reaction environment” as used herein generally refers to a volume of space in which a reaction can take place typically where reactants are at least temporarily contained or confined allowing for detection of at least one reaction product. Examples of a reaction environment include but are not limited to cuvettes, tubes, bottles, as well as one or more depressions, wells, or chambers on a planar or non-planar substrate.

The term “virtual terminator” as used herein generally refers to terminators substantially slow reaction kinetics where additional steps may be employed to stop the reaction such as the removal of reactants.

Some exemplary embodiments of systems and methods associated with sample preparation and processing, generation of sequence data, and analysis of sequence data are generally described below, some or all of which are amenable for use with embodiments of the presently described invention. In particular, the exemplary embodiments of systems and methods for preparation of template nucleic acid molecules, amplification of template molecules, generating target specific amplicons and/or genomic libraries, sequencing methods and instrumentation, and computer systems are described.

In typical embodiments, the nucleic acid molecules derived from an experimental or diagnostic sample should be prepared and processed from its raw form into template molecules amenable for high throughput sequencing. The processing methods may vary from application to application, resulting in template molecules comprising various characteristics. For example, in some embodiments of high throughput sequencing, it is preferable to generate template molecules with a sequence or read length that is at least comparable to the length that a particular sequencing method can accurately produce sequence data for. In the present example, the length may include a range of about 25-30 bases, about 50-100 bases, about 200-300 bases, about 350-500 bases, about 500-1000 bases, greater than 1000 bases, or any other length amenable for a particular sequencing application. In some embodiments, nucleic acids from a sample, such as a genomic sample, are fragmented using a number of methods known to those of ordinary skill in the art. In preferred embodiments, methods that randomly fragment (i.e. do not select for specific sequences or regions) nucleic acids and may include what is referred to as nebulization or sonication methods. It will, however, be appreciated that other methods of fragmentation, such as digestion using restriction endonucleases, may be employed for fragmentation purposes. Also in the present example, some processing methods may employ size selection methods known in the art to selectively isolate nucleic acid fragments of the desired length.

Also, it is preferable in some embodiments to associate additional functional elements with each template nucleic acid molecule. The elements may be employed for a variety of functions including, but not limited to, primer sequences for amplification and/or sequencing methods, quality control elements (i.e. such as Key elements or other type of quality control element), unique identifiers (also referred to as a multiplex identifier or “MID”) that encode various associations such as with a sample of origin or patient, or other functional element.

For example, some embodiments of the described invention comprise associating one or more embodiments of an MID element having a known and identifiable sequence composition with a sample, and coupling the embodiments of MID element with template nucleic acid molecules from the associated samples. The MID coupled template nucleic acid molecules from a number of different samples are pooled into a single “Multiplexed” sample or composition that can then be efficiently processed to produce sequence data for each MID coupled template nucleic acid molecule. The sequence data for each template nucleic acid is de-convoluted to identify the sequence composition of coupled MID elements and association with sample of origin identified. In the present example, a multiplexed composition may include representatives from about 384 samples, about 96 samples, about 50 samples, about 20 samples, about 16 samples, about 12 samples, about 10 samples, or other number of samples. Each sample may be associated with a different experimental condition, treatment, species, or individual in a research context. Similarly, each sample may be associated with a different tissue, cell, individual, condition, drug or other treatment in a diagnostic context. Those of ordinary skill in the related art will appreciate that the numbers of samples listed above are provided for exemplary purposes and thus should not be considered limiting.

In preferred embodiments, the sequence composition of each MID element is easily identifiable and resistant to introduced error from sequencing processes. Some embodiments of MID element comprise a unique sequence composition of nucleic acid species that has minimal sequence similarity to a naturally occurring sequence. Alternatively, embodiments of a MID element may include some degree of sequence similarity to naturally occurring sequence.

Also, in preferred embodiments, the position of each MID element is known relative to some feature of the template nucleic acid molecule and/or adaptor elements coupled to the template molecule. Having a known position of each MID is useful for finding the MID element in sequence data and interpretation of the MID sequence composition for possible errors and subsequent association with the sample of origin.

For example, some features useful as anchors for positional relationship to MID elements may include, but are not limited to, the length of the template molecule (i.e. the MID element is known to be so many sequence positions from the 5′ or 3′ end), recognizable sequence markers such as a Key element and/or one or more primer elements positioned adjacent to a MID element. In the present example, the Key and primer elements generally comprise a known sequence composition that typically does not vary from sample to sample in the multiplex composition and may be employed as positional references for searching for the MID element. An analysis algorithm implemented by application 135 may be executed on computer 130 to analyze generated sequence data for each MID coupled template to identify the more easily recognizable Key and/or primer elements, and extrapolate from those positions to identify a sequence region presumed to include the sequence of the MID element. Application 135 may then process the sequence composition of the presumed region and possibly some distance away in the flanking regions to positively identify the MID element and its sequence composition.

Some or all of the described functional elements may be combined into adaptor elements that are coupled to nucleotide sequences in certain processing steps. For example, some embodiments may associate priming sequence elements or regions comprising complementary sequence composition to primer sequences employed for amplification and/or sequencing. Further, the same elements may be employed for what may be referred to as “strand selection” and immobilization of nucleic acid molecules to a solid phase substrate. In some embodiments, two sets of priming sequence regions (hereafter referred to as priming sequence A, and priming sequence B) may be employed for strand selection, where only single strands having one copy of priming sequence A and one copy of priming sequence B is selected and included as the prepared sample. In alternative embodiments, design characteristics of the adaptor elements eliminate the need for strand selection. The same priming sequence regions may be employed in methods for amplification and immobilization where, for instance, priming sequence B may be immobilized upon a solid substrate and amplified products are extended therefrom.

Additional examples of sample processing for fragmentation, strand selection, and addition of functional elements and adaptors are described in U.S. patent application Ser. No. 10/767,894, titled “Method for preparing single-stranded DNA libraries”, filed Jan. 28, 2004; U.S. patent application Ser. No. 12/156,242, titled “System and Method for Identification of Individual Samples from a Multiplex Mixture”, filed May 29, 2008; and U.S. patent application Ser. No. 12/380,139, titled “System and Method for Improved Processing of Nucleic Acids for Production of Sequencable Libraries”, filed Feb. 23, 2009, each of which is hereby incorporated by reference herein in its entirety for all purposes.

Various examples of systems and methods for performing amplification of template nucleic acid molecules to generate populations of substantially identical copies are described. It will be apparent to those of ordinary skill that it is desirable in some embodiments of SBS to generate many copies of each nucleic acid element to generate a stronger signal when one or more nucleotide species is incorporated into each nascent molecule associated with a copy of the template molecule. There are many techniques known in the art for generating copies of nucleic acid molecules such as, for instance, amplification using what are referred to as bacterial vectors, “Rolling Circle” amplification (described in U.S. Pat. Nos. 6,274,320 and 7,211,390, incorporated by reference above) and Polymerase Chain Reaction (PCR) methods, each of the techniques are applicable for use with the presently described invention. One PCR technique that is particularly amenable to high throughput applications include what are referred to as emulsion PCR methods (also referred to as emPCR methods).

Typical embodiments of emulsion PCR methods include creating a stable emulsion of two immiscible substances creating aqueous droplets within which reactions may occur. In particular, the aqueous droplets of an emulsion amenable for use in PCR methods may include a first fluid, such as a water based fluid suspended or dispersed as droplets (also referred to as a discontinuous phase) within another fluid, such as a hydrophobic fluid (also referred to as a continuous phase) that typically includes some type of oil. Examples of oil that may be employed include, but are not limited to, mineral oils, silicone based oils, or fluorinated oils.

Further, some emulsion embodiments may employ surfactants that act to stabilize the emulsion, which may be particularly useful for specific processing methods such as PCR. Some embodiments of surfactant may include one or more of a silicone or fluorinated surfactant. For example, one or more non-ionic surfactants may be employed that include, but are not limited to, sorbitan monooleate (also referred to as Span 80), polyoxyethylenesorbitsan monooleate (also referred to as Tween 80), or in some preferred embodiments, dimethicone copolyol (also referred to as Abil EM90), polysiloxane, polyalkyl polyether copolymer, polyglycerol esters, poloxamers, and PVP/hexadecane copolymers (also referred to as Unimer U-151), or in more preferred embodiments, a high molecular weight silicone polyether in cyclopentasiloxane (also referred to as DC 5225C available from Dow Corning).

The droplets of an emulsion may also be referred to as compartments, microcapsules, microreactors, microenvironments, or other name commonly used in the related art. The aqueous droplets may range in size depending on the composition of the emulsion components or composition, contents contained therein, and formation technique employed. The described emulsions create the microenvironments within which chemical reactions, such as PCR, may be performed. For example, template nucleic acids and all reagents necessary to perform a desired PCR reaction may be encapsulated and chemically isolated in the droplets of an emulsion. Additional surfactants or other stabilizing agent may be employed in some embodiments to promote additional stability of the droplets as described above. Thermocycling operations typical of PCR methods may be executed using the droplets to amplify an encapsulated nucleic acid template resulting in the generation of a population comprising many substantially identical copies of the template nucleic acid. In some embodiments, the population within the droplet may be referred to as a “clonally isolated”, “compartmentalized”, “sequestered”, “encapsulated”, or “localized” population. Also in the present example, some or all of the described droplets may further encapsulate a solid substrate such as a bead for attachment of template and amplified copies of the template, amplified copies complementary to the template, or combination thereof. Further, the solid substrate may be enabled for attachment of other type of nucleic acids, reagents, labels, or other molecules of interest.

After emulsion breaking and bead recovery, it may also be desirable in typical embodiments to “enrich” for beads having a successfully amplified population of substantially identical copies of a template nucleic acid molecule immobilized thereon. For example, a process for enriching for “DNA positive” beads may include hybridizing a primer species to a region on the free ends of the immobilized amplified copies, typically found in an adaptor sequence, extending the primer using a polymerase mediated extension reaction, and binding the primer to an enrichment substrate such as a magnetic or sepharose bead. A selective condition may be applied to the solution comprising the beads, such as a magnetic field or centrifugation, where the enrichment bead is responsive to the selective condition and is separated from the “DNA negative” beads (i.e. NO: or few immobilized copies).

Embodiments of an emulsion useful with the presently described invention may include a very high density of droplets or microcapsules enabling the described chemical reactions to be performed in a massively parallel way. Additional examples of emulsions employed for amplification and their uses for sequencing applications are described in U.S. Pat. Nos. 7,638,276; 7,622,280; 7,842,457; 7,927,797; and 8,012,690 and U.S. patent application Ser. No. 13/033,240, each of which is hereby incorporated by reference herein in its entirety for all purposes.

Also embodiments sometimes referred to as Ultra-Deep Sequencing, generate target specific amplicons for sequencing may be employed with the presently described invention that include using sets of specific nucleic acid primers to amplify a selected target region or regions from a sample comprising the target nucleic acid. Further, the sample may include a population of nucleic acid molecules that are known or suspected to contain sequence variants comprising sequence composition associated with a research or diagnostic utility where the primers may be employed to amplify and provide insight into the distribution of sequence variants in the sample. For example, a method for identifying a sequence variant by specific amplification and sequencing of multiple alleles in a nucleic acid sample may be performed. The nucleic acid is first subjected to amplification by a pair of PCR primers designed to amplify a region surrounding the region of interest or segment common to the nucleic acid population. Each of the products of the PCR reaction (first amplicons) is subsequently further amplified individually in separate reaction vessels such as an emulsion based vessel described above. The resulting amplicons (referred to herein as second amplicons), each derived from one member of the first population of amplicons, are sequenced and the collection of sequences are used to determine an allelic frequency of one or more variants present. Importantly, the method does not require previous knowledge of the variants present and can typically identify variants present at <1% frequency in the population of nucleic acid molecules.

Some advantages of the described target specific amplification and sequencing methods include a higher level of sensitivity than previously achieved and are particularly useful for strategies comprising mixed populations of template nucleic acid molecules. Further, embodiments that employ high throughput sequencing instrumentation, such as for instance embodiments that employ what is referred to as a PicoTiterPlate array (also sometimes referred to as a PTP plate or array) of wells provided by 454 Life Sciences Corporation, the described methods can be employed to generate sequence composition for over 100,000, over 300,000, over 500,000, or over 1,000,000 nucleic acid regions per run or experiment and may depend, at least in part, on user preferences such as lane configurations enabled by the use of gaskets, etc. Also, the described methods provide a sensitivity of detection of low abundance alleles which may represent 1% or less of the allelic variants present in a sample. Another advantage of the methods includes generating data comprising the sequence of the analyzed region. Importantly, it is not necessary to have prior knowledge of the sequence of the locus being analyzed.

Additional examples of target specific amplicons for sequencing are described in U.S. patent application Ser. No. 11/104,781, titled “Methods for determining sequence variants using ultra-deep sequencing”, filed Apr. 12, 2005; PCT Patent Application Serial No. US 2008/003424, titled “System and Method for Detection of HIV Drug Resistant Variants”, filed Mar. 14, 2008; and U.S. Pat. No. 7,888,034, titled “System and Method for Detection of HIV Tropism Variants”, filed Jun. 17, 2009; and U.S. patent application Ser. No. 12/592,243, titled “SYSTEM AND METHOD FOR DETECTION OF HIV INTEGRASE VARIANTS”, filed Nov. 19, 2009, each of which is hereby incorporated by reference herein in its entirety for all purposes.

Further, embodiments of sequencing may include Sanger type techniques, techniques generally referred to as Sequencing by Hybridization (SBH), Sequencing by Ligation (SBL), or Sequencing by Incorporation (SBI) techniques. The sequencing techniques may also include what are referred to as polony sequencing techniques; nanopore, waveguide and other single molecule detection techniques; or reversible terminator techniques. As described above, a preferred technique may include Sequencing by Synthesis methods. For example, some SBS embodiments sequence populations of substantially identical copies of a nucleic acid template and typically employ one or more oligonucleotide primers designed to anneal to a predetermined, complementary position of the sample template molecule or one or more adaptors attached to the template molecule. The primer/template complex is presented with a nucleotide species in the presence of a nucleic acid polymerase enzyme. If the nucleotide species is complementary to the nucleic acid species corresponding to a sequence position on the sample template molecule that is directly adjacent to the 3′ end of the oligonucleotide primer, then the polymerase will extend the primer with the nucleotide species. Alternatively, in some embodiments the primer/template complex is presented with a plurality of nucleotide species of interest (typically A, G, C, and T) at once, and the nucleotide species that is complementary at the corresponding sequence position on the sample template molecule directly adjacent to the 3′ end of the oligonucleotide primer is incorporated. In either of the described embodiments, the nucleotide species may be chemically blocked (such as at the 3′-O position) to prevent further extension, and need to be deblocked prior to the next round of synthesis. It will also be appreciated that the process of adding a nucleotide species to the end of a nascent molecule is substantially the same as that described above for addition to the end of a primer.

As described above, incorporation of the nucleotide species can be detected by a variety of methods known in the art, e.g. by detecting the release of pyrophosphate (PPi) using an enzymatic reaction process to produce light or via detection the release of H⁺ and measurement of pH change (examples described in U.S. Pat. Nos. 6,210,891; 6,258,568; and 6,828,100, each of which is hereby incorporated by reference herein in its entirety for all purposes), or via detectable labels bound to the nucleotides. Some examples of detectable labels include, but are not limited to, mass tags and fluorescent or chemiluminescent labels. In typical embodiments, unincorporated nucleotides are removed, for example by washing. Further, in some embodiments, the unincorporated nucleotides may be subjected to enzymatic degradation such as, for instance, degradation using the apyrase or pyrophosphatase enzymes as described in U.S. patent application Ser. No. 12/215,455, titled “System and Method for Adaptive Reagent Control in Nucleic Acid Sequencing”, filed Jun. 27, 2008; and Ser. No. 12/322,284, titled “System and Method for Improved Signal Detection in Nucleic Acid Sequencing”, filed Jan. 29, 2009; each of which is hereby incorporated by reference herein in its entirety for all purposes.

In the embodiments where detectable labels are used, they will typically have to be inactivated (e.g. by chemical cleavage or photobleaching) prior to the following cycle of synthesis. The next sequence position in the template/polymerase complex can then be queried with another nucleotide species, or a plurality of nucleotide species of interest, as described above. Repeated cycles of nucleotide addition, extension, signal acquisition, and washing result in a determination of the nucleotide sequence of the template strand. Continuing with the present example, a large number or population of substantially identical template molecules (e.g. 10³, 10⁴, 10⁵, 10⁶ or 10⁷ molecules) are typically analyzed simultaneously in any one sequencing reaction, in order to achieve a signal which is strong enough for reliable detection.

In addition, it may be advantageous in some embodiments to improve the read length capabilities and qualities of a sequencing process by employing what may be referred to as a “paired-end” sequencing strategy. For example, some embodiments of sequencing method have limitations on the total length of molecule from which a high quality and reliable read may be generated. In other words, the total number of sequence positions for a reliable read length may not exceed 25, 50, 100, or 500 bases depending on the sequencing embodiment employed. A paired-end sequencing strategy extends reliable read length by separately sequencing each end of a molecule (sometimes referred to as a “tag” end) that comprise a fragment of an original template nucleic acid molecule at each end joined in the center by a linker sequence. The original positional relationship of the template fragments is known and thus the data from the sequence reads may be re-combined into a single read having a longer high quality read length. Further examples of paired-end sequencing embodiments are described in U.S. Pat. No. 7,601,499, titled “Paired end sequencing”; and in U.S. patent application Ser. No. 12/322,119, titled “Paired end sequencing”, filed Jan. 28, 2009, each of which is hereby incorporated by reference herein in its entirety for all purposes.

Some examples of SBS apparatus may implement some or all of the methods described above and may include one or more of a detection device such as a charge coupled device (i.e., CCD camera) or confocal type architecture for optical detection, Ion-Sensitive Field Effect Transistor (also referred to as “ISFET”) or Chemical-Sensitive Field Effect Transistor (also referred to as “ChemFET”) for architectures for ion or chemical detection, a microfluidics chamber or flow cell, a reaction substrate, and/or a pump and flow valves. Taking the example of pyrophosphate-based sequencing, some embodiments of an apparatus may employ a chemiluminescent detection strategy that produces an inherently low level of background noise.

In some embodiments, the reaction substrate for sequencing may include a planar substrate, such as a slide type substrate, a semiconductor chip comprising well type structures with ISFET detection elements contained therein, or waveguide type reaction substrate that in some embodiments may comprise well type structures. Further, the reaction substrate may include what is referred to as a PTP array available from 454 Life Sciences Corporation, as described above, formed from a fiber optic faceplate that is acid-etched to yield hundreds of thousands or more of very small wells each enabled to hold a population of substantially identical template molecules (i.e., some preferred embodiments comprise about 3.3 million wells on a 70×75 mm PTP array at a 35 well to well pitch). In some embodiments, each population of substantially identical template molecule may be disposed upon a solid substrate, such as a bead, each of which may be disposed in one of said wells. For example, an apparatus may include a reagent delivery element for providing fluid reagents to the PTP plate holders, as well as a CCD type detection device enabled to collect photons of light emitted from each well on the PTP plate. An example of reaction substrates comprising characteristics for improved signal recognition is described in U.S. Pat. No. 7,682,816, titled “THIN-FILM COATED MICROWELL ARRAYS AND METHODS OF MAKING SAME”, filed Aug. 30, 2005, which is hereby incorporated by reference herein in its entirety for all purposes. Further examples of apparatus and methods for performing SBS type sequencing and pyrophosphate sequencing are described in U.S. Pat. Nos. 7,323,305 and 7,575,865, both of which are incorporated by reference above.

In addition, systems and methods may be employed that automate one or more sample preparation processes, such as the emPCR process described above. For example, automated systems may be employed to provide an efficient solution for generating an emulsion for emPCR processing, performing PCR Thermocycling operations, and enriching for successfully prepared populations of nucleic acid molecules for sequencing. Examples of automated sample preparation systems are described in U.S. Pat. No. 7,927,797; and U.S. patent application Ser. No. 13/045,210, each of which is hereby incorporated by reference herein in its entirety for all purposes.

Also, the systems and methods of the presently described embodiments of the invention may include implementation of some design, analysis, or other operation using a computer readable medium stored for execution on a computer system. For example, several embodiments are described in detail below to process detected signals and/or analyze data generated using SBS systems and methods where the processing and analysis embodiments are implementable on computer systems.

In some embodiments a data processing application includes algorithms for correcting raw sequence data for the accumulations of CAFIE error. For example, some or all of the CAIFE error factors may be accurately approximated and applied to a theoretical flowgram model to provide a representation of real data obtained from an actual sequencing run and subsequently approximate a theoretical flowgram from an observed flowgram using an inversion of a mathematical model. Thus, an approximation of error may be applied to actual sequencing data represented in an observed flowgram to produce a theoretical flowgram representing the sequence composition of a target nucleic acid with all or substantially all of the error factors removed. Additional examples of CAFIE correction embodiments are described in U.S. Pat. Nos. 8,301,394; and 8,364,417, each of which are hereby incorporated by reference herein in its entirety for all purposes.

An exemplary embodiment of a computer system for use with the presently described invention may include any type of computer platform such as a workstation, a personal computer, a server, or any other present or future computer. It will, however, be appreciated by one of ordinary skill in the art that the aforementioned computer platforms as described herein are specifically configured to perform the specialized operations of the described invention and are not considered general purpose computers. Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices.

Display devices may include display devices that provide visual information, this information typically may be logically and/or physically organized as an array of pixels. An interface controller may also be included that may comprise any of a variety of known or future software programs for providing input and output interfaces. For example, interfaces may include what are generally referred to as “Graphical User Interfaces” (often referred to as GUI's) that provides one or more graphical representations to a user. Interfaces are typically enabled to accept user inputs using means of selection or input known to those of ordinary skill in the related art.

In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUI's, CLI's or a combination thereof.

A processor may include a commercially available processor such as a Celeron, Core, or Pentium processor made by Intel Corporation, a SPARC processor made by Sun Microsystems, an Athlon, Sempron, Phenom, or Opteron processor made by AMD corporation, or it may be one of other processors that are or will become available. Some embodiments of a processor may include what is referred to as Multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related will appreciate that a processor may be configured in what is generally referred to as 32 or 64 bit architectures, or other architectural configurations now known or that may be developed in the future.

A processor typically executes an operating system, which may be, for example, a Windows-type operating system (such as Windows XP, Windows Vista, or Windows_{—}7) from the Microsoft Corporation; the Mac OS X operating system from Apple Computer Corp. (such as Mac OS X v10.6 “Snow Leopard” operating systems); a Unix or Linux-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

System memory may include any of a variety of known or future memory storage devices. Examples include any commonly available random access memory (RAM), magnetic medium, such as a resident hard disk or tape, an optical medium such as a read and write compact disc, or other memory storage device. Memory storage devices may include any of a variety of known or future devices, including a compact disk drive, a tape drive, a removable hard disk drive, USB or flash drive, or a diskette drive. Such types of memory storage devices typically read from, and/or write to, a program storage medium such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with memory storage device.

In some embodiments, a computer program product is described comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers could include any of a variety of known devices for accepting and processing information from a user, whether a human or a machine, whether local or remote. Such devices include, for example, modem cards, wireless cards, network interface cards, sound cards, or other types of controllers for any of a variety of known input devices. Output controllers could include controllers for any of a variety of known display devices for presenting information to a user, whether a human or a machine, whether local or remote. In the presently described embodiment, the functional elements of a computer communicate with each other via a system bus. Some embodiments of a computer may communicate with some functional elements using network or other types of remote communications.

As will be evident to those skilled in the relevant art, an instrument control and/or a data processing application, if implemented in software, may be loaded into and executed from system memory and/or a memory storage device. All or portions of the instrument control and/or data processing applications may also reside in a read-only memory or similar device of the memory storage device, such devices not requiring that the instrument control and/or data processing applications first be loaded through input-output controllers. It will be understood by those skilled in the relevant art that the instrument control and/or data processing applications, or portions of it, may be loaded by a processor in a known manner into system memory, or cache memory, or both, as advantageous for execution.

Also, a computer may include one or more library files, experiment data files, and an internet client stored in system memory. For example, experiment data could include data related to one or more experiments or assays such as detected signal values, or other values associated with one or more SBS experiments or processes. Additionally, an internet client may include an application enabled to accesses a remote service on another computer using a network and may for instance comprise what are generally referred to as “Web Browsers”. In the present example, some commonly employed web browsers include Microsoft Internet Explorer 8 available from Microsoft Corporation, Mozilla Firefox 3.6 from the Mozilla Corporation, Safari 4 from Apple Computer Corp., Google Chrome from the Google Corporation, or other type of web browser currently known in the art or to be developed in the future. Also, in the same or other embodiments an internet client may include, or could be an element of, specialized software applications enabled to access remote information via a network such as a data processing application for biological applications.

A network may include one or more of the many various types of networks well known to those of ordinary skill in the art. For example, a network may include a local or wide area network that may employ what is commonly referred to as a TCP/IP protocol suite to communicate. A network may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

### b. EMBODIMENTS OF THE PRESENTLY DESCRIBED INVENTION

As described above, embodiments of the described invention relate to a system and method comprising an ISFET based detection platform comprising one or more arrays of wells for sequencing nucleic acid template molecules where one or more wells do not have a species of template nucleic acid, instead having a high pH buffering substrate disposed therein which is employed as a reference well in methods that process signals detected from wells that comprise template nucleic acid. Also, in the same or alternative embodiments high pH buffering substrates may be employed to reduce well to well communication of H⁺ ions.

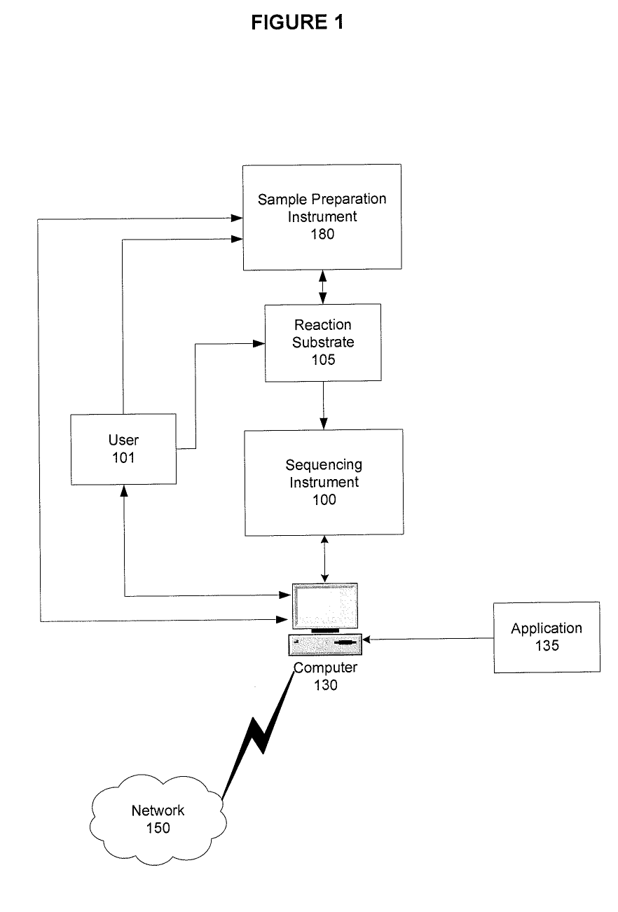

In a typical sequencing embodiment, one or more instrument elements may be employed that automate one or more process steps. For example, embodiments of a sequencing method may be executed using instrumentation to automate and carry out some or all process steps. FIG. 1 provides an illustrative example of sequencing instrument 100 constructed and arranged for sequencing processes requiring capture of signals from one or more embodiments of substrate 105. In some embodiments, reaction substrate 105 comprises a plurality of Ion Sensitive Field Effect Transistors (often referred to as ISFET). Also in the same or alternative embodiments, sequencing instrument 100 comprises a subsystem that operatively couples with substrate 105 with one or more data processing elements, and a fluidic subsystem that enables execution of sequencing reactions on reaction substrate 105. It will, however, be appreciated that for sequencing processes requiring other modes of data capture (i.e. temperature, electric current, electrochemical, etc.), a subsystem for the mode of data capture may be employed which are known to those of ordinary skill in the related art. For instance, a sample of template molecules may be loaded onto reaction substrate 105 by user 101 or some automated embodiment, then sequenced in a massively parallel manner using sequencing instrument 100 to produce sequence data representing the sequence composition of each template nucleic acid molecule. Importantly, user 101 may include any type of user of sequencing technologies.

In some embodiments, samples may be optionally prepared for sequencing in a fully automated or partially automated fashion using sample preparation instrument 180 configured to perform some or all of the necessary sample preparation steps for sequencing using instrument 100. Those of ordinary skill in the art will appreciate that sample preparation instrument 180 is provided for the purposes of illustration and may represent one or more instruments each designed to carry out some or all of the steps associated with sample preparation required for a particular sequencing assay. Examples of sample preparation instruments may include robotic and/or microfluidic platforms such as those available from Hamilton Robotics, Fluidigm Corporation, Beckman Coulter, Agilent Technologies, or Caliper Life Sciences.

Further, as illustrated in FIG. 1, sequencing instrument 100 may be operatively linked to one or more external computer components, such as computer 130 that may, for instance, execute system software or firmware, such as application 135 that may provide instructional control of one or more of the instruments, such as sequencing instrument 100 or sample preparation instrument 180, and/or signal processing/data analysis functions. Computer 130 may be additionally operatively connected to other computers or servers via network 150 that may enable remote operation of instrument systems and the export of large amounts of data to systems capable of storage and processing. Also in some embodiments network 150 may enable what is referred to as “cloud computing” for signal processing and/or data analysis functions. In the present example, sequencing instrument 100 and/or computer 130 may include some or all of the components and characteristics of the embodiments generally described herein.

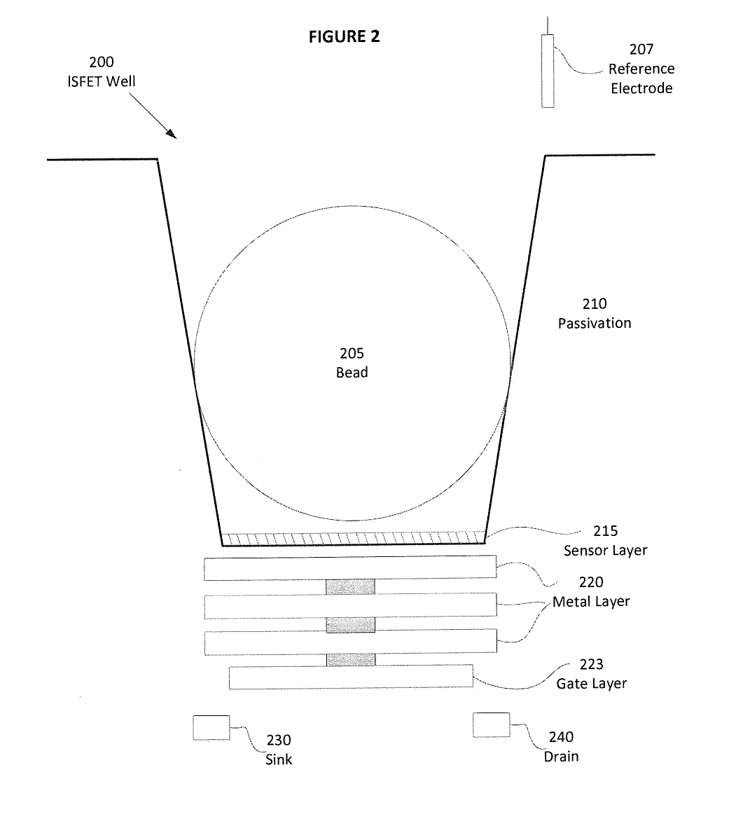

Embodiments on the presently described invention comprise arrays of individual ISFET sensors constructed and arranged to detect minute changes of pH through the ion-exchange between an aqueous solution and a sensing surface associated with each individual ISFET sensor. In some embodiments the ISFET sensors are individually disposed at the bottom surface of well structures that are typically constructed as a planar array of well structures, where each well comprises at least one ISFET sensor. An illustrative example of one possible embodiment of a well structure comprising an ISFET sensor embodiment is provided in FIG. 2 as ISFET well 200. ISFET well 200 comprises a sidewall structure constructed of a passivation material (illustrated as passivation 210), where in addition to the sidewall structure passivation 210 may optionally also comprise a layer of passivation 210 material at the bottom surface of well 200. In embodiments where a layer of passivation 210 is present at the bottom surface it will be appreciated that the passivating material may comprise a different composition than the passivating material of the sidewall structure. It will also be appreciated that FIG. 2 is represented as a 2 dimensional illustration and it should be appreciated that well 200 comprises sidewall structure that fully surrounds and creates a perimeter for well 200 thereby creating physical separation between each embodiment of well 200 in an array of wells that are open at the top for fluid communication within a common flowcell, which in some embodiments comprise an aqueous solution. In FIG. 2, well 200 also includes illustrative examples of embodiments of ISFET structures that include metal layers 220, gate layer 223 (represented as a “floating gate”), as well as sink 230 and drain 240. It will, however, be appreciated that the ISFET structures represented in FIG. 2 are for the purposes of illustration only and should not be construed as limiting.

Also illustrated in FIG. 2 is bead 205 which, as described elsewhere in this specification, may have a population of substantially identical copies of a species of template nucleic acid disposed thereon useful for sequencing methods. Alternatively, bead 205 may comprise a high pH buffering characteristic which will be described in greater detail below in the context of the presently described invention. Well 200 also comprises sensor layer 215 that in the described embodiments provides separation between the gate electrodes (metal layers 220 and/or gate layer 223) and is typically sensitive to Hydrogen ions (H⁺) as well as insensitive to salt. Reference electrode 207 is also illustrated in FIG. 2 as being physically located outside of but in fluid communication with well 200.

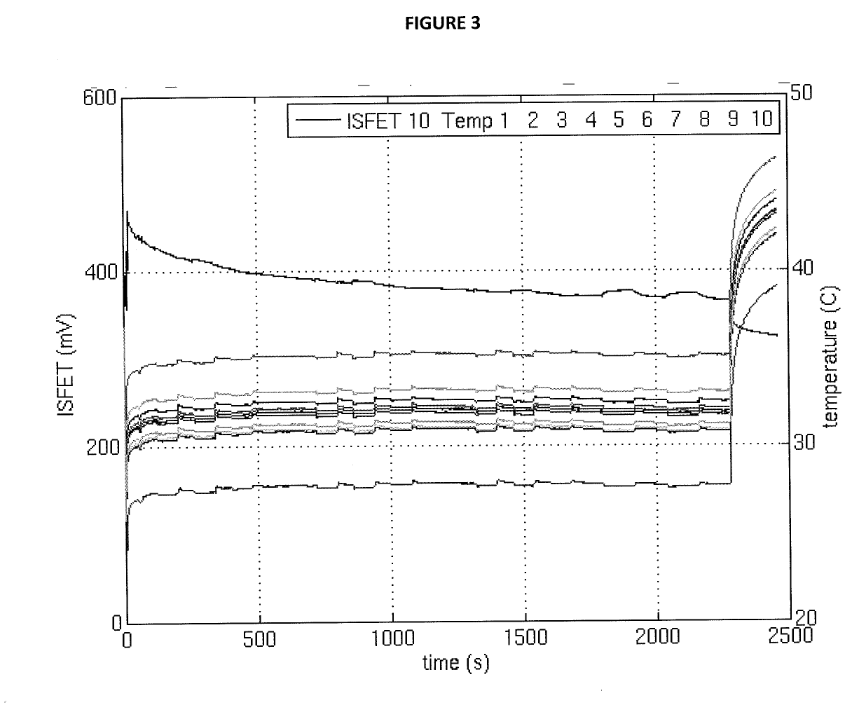

In the presently described embodiments, a change in pH of the aqueous solution within well 200, such as what locally occurs upon successful incorporation of a nucleotide species in a sequencing by synthesis reaction, results in a corresponding change in the surface potential of sensing layer 215 that is converted to electrical signals by the FET structure underneath. In practice, the ISFET sensors not only respond to the change of pH but are also sensitive to other sources that cause surface potential changes of sensing layer 215 such as temperature changes and electrical signal pickup from the surrounding environment. An example of ISFET detection of temperature generated signals is provided in the illustrative example of FIG. 3 that shows differences in detected levels of signal (in mV) by an ISFET sensor over a signal acquisition time period at different temperatures (in ° C.).

In the described embodiments the signals generated from the sources not related to pH change create “noise” in the total signal detected by the ISFET that can make it very difficult to discriminate very small or rapid signal changes associated with a pH change. Therefore it is desirable to have a plurality of reference sensors distributed across the array that are inert to changes in pH but are electrically connected to the same embodiment of reference electrode 207 as the sensors sensitive to pH, so that signals from the reference sensors are substantially generated from noise sources only.

As those of ordinary skill in the art appreciate, a differential measurement between a pH sensitive ISFET sensor and a reference ISFET sensor can be employed in a signal processing method, such as what may be executed by application 135, to effectively eliminate the noise signal (combined signal from all non-pH sources) that results in a signal substantially associated with a pH change in the aqueous solution only. This method is typically referred to as Reference FET (REFET). The following equations summarize the principle of using a REFET to extract the pH response from an ISFET.

ISFET response=pH response+thermal response+electrical noise+other non-pH response

REFET response=thermal response+electrical noise+other non-pH response Differential measurement=ISFET response−REFET response=pH response

Conventional methods for implementing REFET typically involve blocking the ion exchange between the aqueous fluid and the sensing surface by either chemically modifying or physically blocking the sensing surface so that the ions do not make direct contact. For example, casting a PVC membrane on the sensing surface has been employed to reduce the pH response of FET sensors. However, these methods increase the complexity of fabricating arrays of ISFET sensors and do not completely eliminate pH responses. More importantly, they pose the risk of electrically disconnecting the sensing surface of the reference sensor and reference electrode.

Embodiments of the presently described invention overcome these difficulties in an inexpensive and simple manner. For example, reference sensors can be created by depositing beads with high pH buffering capacity to wells of an ISFET sensor array. As those of ordinary skill in the art appreciate, a pH buffer can accept or donate H⁺ ions depending on the pH level of the solution it is in and moderates changes in pH by donating H⁺ when in a more basic solution than its buffering equilibrium and sequestering H⁺ when in a more acidic solution. The use of pH buffers in the presently described invention are useful to reduce or remove the effects of sudden changes in pH providing essentially a steady state pH condition.

One example of beads with high pH buffering capacity includes beads that are typically composed on non-buffering materials such as PEG, polystyrene, or other type of non pH buffering material known in the art, and having a high degree of porosity. In the described embodiments the pH buffering characteristics are provided by functionalizing the beads with groups that buffer pH in a range of 7-8. Examples of functional groups include phosphonates, hydroxamic acid, amino acid, and carboxylic acid functional groups attached to the surface areas of the beads. It will be appreciated that the measure of porosity of the bead substrate contributes to the amount of surface area available for functionalization where, for instance, a high degree of porosity provides a high degree of available surface area relative to a bead of similar dimension having a low degree of porosity. Those of ordinary skill in the art also appreciate that nucleic acid molecules, being acids, also possess some pH buffering characteristics and that beads comprising populations of nucleic acid template species will buffer pH to some degree. However, in the presently described embodiments the high pH buffering beads described herein have substantially higher and easily distinguishable pH buffering characteristics than do the described nucleic acid template beads. It will also be appreciated that there are many types of functional groups and molecules that can act as a pH buffer that include but are not limited to bicarbonate based buffers, phosphate based buffers, proteins, and nucleic acids.

In some embodiments the high buffering beads may be combined with beads comprising the populations of nucleic acid template species and randomly distributed over the array of wells resulting in a proportion of wells comprising only the high buffering beads in close proximity to the sensing surfaces associated with the ISFET sensors. Alternatively, the high buffering beads and template beads may be distributed in a serial fashion to provide a greater degree of control over the spatial distribution and/or relative percentage of wells occupied by particular bead type.

Due to the characteristics of the high buffering beads, substantially any pH changes in the local proximity to the buffering beads, such as the interior space defined by the well structures, are eliminated by the pH buffering characteristics of the high buffering beads. Therefore, in the wells comprising the pH buffering beads the ion exchange between the fluid and the sensing surface is unimpeded and the surface is still sensitive to pH changes, but the pH inside the well is kept constant by the high buffering bead material. Effectively, a pH-inert reference sensor is created. Electrical connection to the reference electrode is guaranteed due to fact that the high buffering beads do not block fluid communication between the sensing surface and reference electrode. In the same or alternative embodiments, some proportion of pH buffering beads may not settle into a well but are positioned substantially outside of the wells sitting on top of a bead within a well or on a wall structure of wells of the planar array, where the pH buffering characteristics inhibits the communication of H⁺ between the well structures.

As those of ordinary skill in the art appreciate buffering capacity, or the ability of resist pH changes, of a material is characterized by its acid dissociation constant, often referred to as the pKa value, which is defined as

$$Ka = \frac{⌊ {A}^{-} ⌋ ⌊ {H}^{+} ⌋}{[ HA ]} , \\ pKa = - {log}_{10} Ka$$

with the acid-base equilibrium defined by the following equation

HA [symbol] H⁺+A⁻

Where HA is the acid, A⁻ is the conjugate base and H⁺ is the hydrogen ion. To be an effective buffer, the pKa value of the material should optimally be in close range to the operating pH value of the fluid, for example±1.

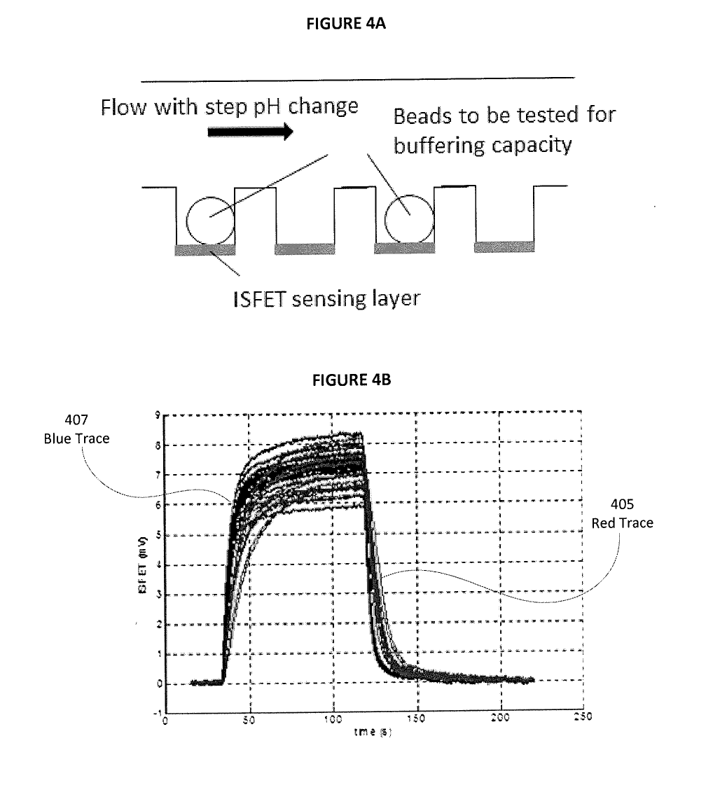

An embodiment of a method for measuring the buffering capacity of the materials confined by the well structure of an ISFET array is described herein where the array includes a plurality of empty wells and a plurality of wells that comprise at least one bead species to be tested for its pH buffering capacity, an illustrative example of which is provided in FIG. 4A. The described embodiment of the method comprises iteratively flowing an ionic solution with a known buffering capacity and a step pH change from the previous flow over an array of wells comprising ISFET sensors and measuring the corresponding ISFET signals from the individual wells for the respective pH level and plotting the measured signals over time. For example, the iterations may include a 0 μM, 10 μM, 100 μM, and 1000 μM concentrations of tris(hydroxymethyl)aminomethane (also referred to as TRIS) solution with stepwise pH changes of pH 7.5, 6.5, and 7.5. In the presently described example, pH buffer capacity of the bead embodiment can be calculated from the Tris concentration and solution introducing the pH change, where low Tris concentrations elicit very little pH response from the ISFET sensors in wells comprising high pH buffering beads.

Those of ordinary skill in the art appreciate that the term transit time (also referred to as the rise time) as used herein generally refers to the rate of change of the detected ISFET signal value in response to the introduction of a detectable element, and in the current example it is the rise time in response to the introduction of the step change in solution pH in combination with the buffering capacity of the material in the well. By nature, a high buffering material resists pH change through hydrogen ion exchange with a buffering species. Consequently, the higher the buffering capacity of the material means that more ions become engaged with the buffering species and do not reach the ISFET or other pH sensor, and results in a longer the rise time to the newly changed/introduced pH value. FIG. 4B provides an example of measured bead buffering capacity by the difference in rise times from the ISFET sensors in the wells with pH buffering beads (Red traces 405) and without (Blue traces 407) pH buffering beads, where the Red traces 405 require more time to reach their maximum detected value that are also smaller than the values associated with the Blue traces 407.

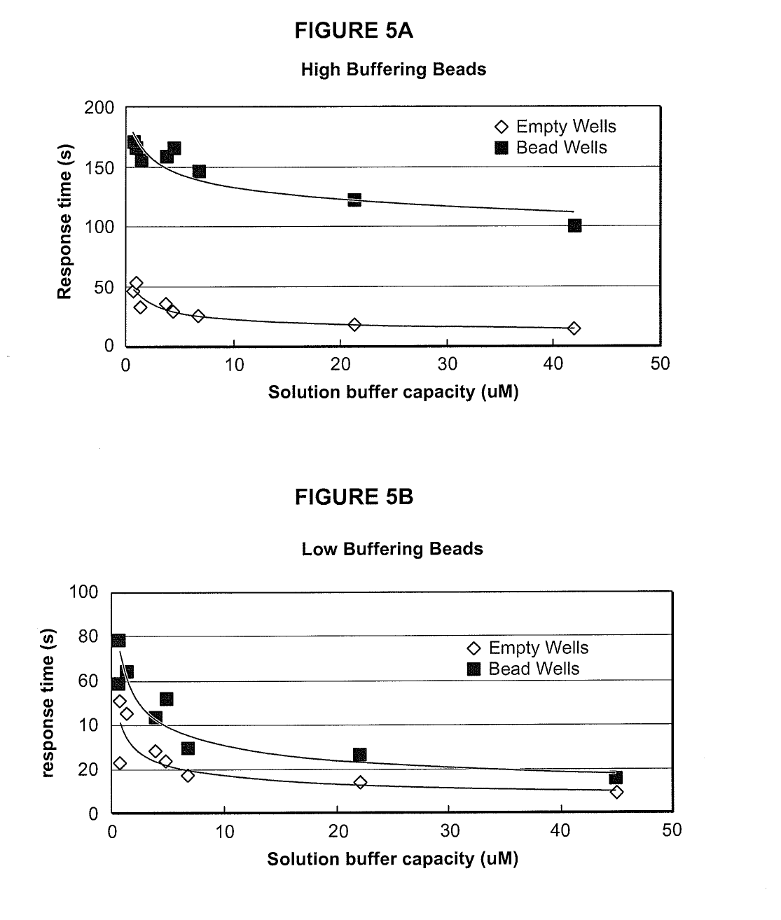

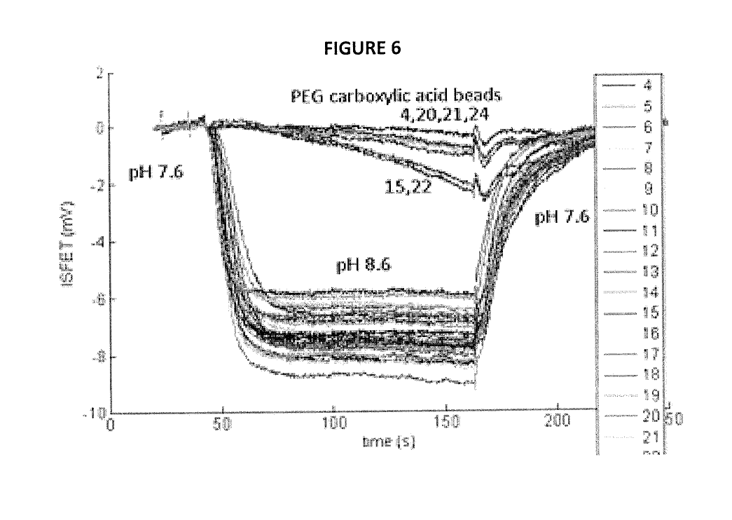

As described above, rise times associated with the beads are measured with solutions with different buffering capacities that can be adjusted by the adding elements/solutions to alter pH buffering characteristics to the measuring solution, such as TRIS. It is generally advantageous to use multiple points of measurement which provides a higher degree of confidence over a single point of measurement, and in the described embodiments the measured rise times may not be sensitive to the pH buffering capacity of the buffering bead embodiments at certain TRIS concentrations. Additional illustrative examples comparing the rise times from high buffering beads to low buffering beads are presented in FIGS. 5A and 5B. The beads with longer rise time illustrated in FIG. 5A have a higher buffering capacity than the beads shown in FIG. 5B, since the higher buffering beads provide better resistance the pH change in the wells. Further, FIG. 6 provides an illustrative example of Polyethylene glycol (PEG) beads functionalized with carboxylic acid placed in wells of an array of 40 wells that, as described above, are capable of buffering substantially all rapid pH changes in a pH range of 7-9, thus creating reference channels which are useful for background subtraction. Each line in the example of FIG. 6 represents a response from an ISFET sensor in a single well, where wells 4, 15, 20, 21, 22, and 24 are wells that comprise a single carboxylic acid functionalized bead with all other wells being empty wells (i.e. no bead substrates).

Typically, a good high buffering material is composed of (1) functional groups with pKa close to the operating pH of the system, and (2) high density of those functional groups. The high buffering material can be in the form of beads which can be deposited into the wells via gravity, magnetic field, centrifugation, or other means for depositing beads into wells known in the art. In the same or alternative embodiments, pH buffering functional groups can be coated on top of a subset of the ISFET sensors during the fabrication process with the help of a mask, so that the locations of the reference sensors are pre-defined and may be used directly in the noise subtraction methods. For example, in one embodiment of the described invention pH buffering functional groups may be employed with arrays of ISFET sensors that have no well structures, where pH buffering functional groups as described above may be immobilized directly onto the surface above one or more ISFET sensors which confer the pH buffering characteristic to those sensors. The sensors associated with the pH buffering functional groups could then be employed as REFETs for other ISFET sensors not associated with any pH buffering functional groups and used for pH detection.

It will also be appreciated that the more optimal functional groups employed for buffering typically act to temporarily sequester H⁺ ions rather than binding H⁺ permanently which would generally result in an eventual saturation condition over the course of a sequencing run. Alternatively, the H⁺ binding would occur for a sufficient duration so that the H⁺ ions are sequestered during the signal acquisition time periods but subsequently released during wash cycles or other cycles where signal detection does not occur and essentially purged from the flow cell environment by the flow through nature of the flow cell.

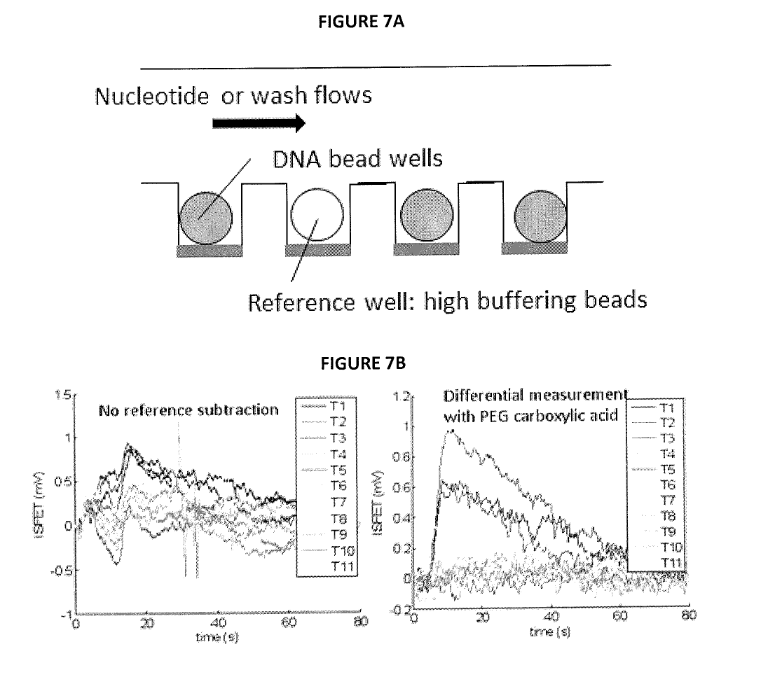

In the described embodiments where pH buffering functional groups are associated with beads, the reference sensors created by high buffering beads are useful for noise subtraction to facilitate the extraction of the incorporation signals from wells comprising nucleic acid template species. In some embodiments, high buffering beads without nucleic acid template are first deposited onto an ISFET array with well structures, followed by the deposition of nucleic acid template beads that may also in some cases have polymerase bound to at least some strands and optionally non-buffering packing bead species into the wells. The high buffering beads can also be mixed with nucleic acid template beads in a single bead deposition. An example of the result achieved using either deposition approach is graphically depicted in FIG. 7A.

In some embodiments, the reference sensors can be initially identified by flowing solutions with a step pH change to the flow cell. The ISFET sensors that do not respond to the expected step pH change are identified as the reference sensors containing high buffering beads. The example in FIG. 7B shows the signals detected from a well comprising a nucleic acid template bead before and after differential measurement by subtracting detected signals from a well containing a high buffer bead. The existence of the reference sensor allows noise removal, which leads to accurate extraction of DNA incorporation signals. More specifically, in FIG. 7B ISFET signals (measured in mV) from a well containing a nucleic acid template bead were detected over the dTTP flows in the first 11 flow cycles over a signal acquisition time period. In the graph on the Left side of FIG. 7B, raw signals comprise a substantial contribution from real time non-repeating noise, and on the Right side, differential measurement with respect to a reference sensor created by high buffer beads reveals the signals detected from the nucleotides species incorporation where a substantial portion of the real time non-repeating noise is removed.

It will be appreciated by those of ordinary skill in the related art that the utility for use of high pH buffering beads to create REFET sensors extends beyond nucleic acid sequencing applications and have use in any pH detection application that uses a plurality of wells, or other means of capture of the beads, that are individually associated with a FET sensor. Examples of other such pH detection applications useful with the embodiments described herein are described in P. Bergveld et al., “How electrical and chemical requirements for refets may coincide,” Sensors and Actuators 18, no. 3-4 (July 1989): 309-327; and A. Errachid, J. Bausells, and N. Jaffrezic-Renault, “A simple REFET for pH detection in differential mode,” Sensors and Actuators B: Chemical 60, no. 1 (Nov. 2, 1999): 43-48, each of which is hereby incorporated by reference herein in its entirety for all purposes.

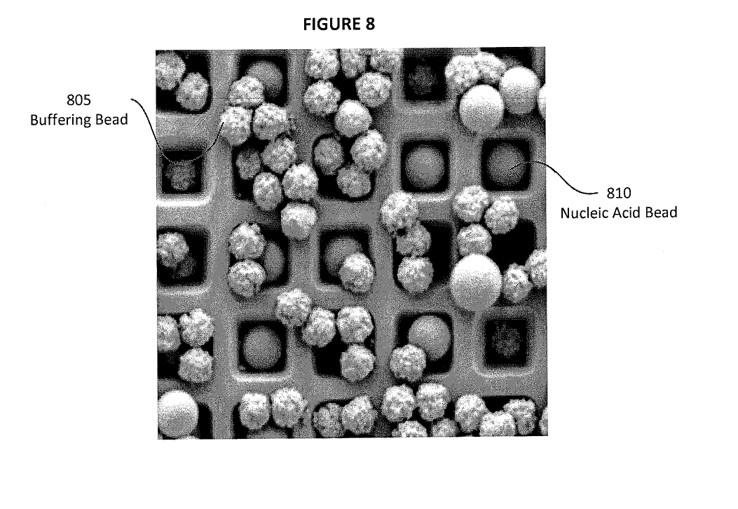

As described above, in some of the described embodiments the use of high pH buffering beads can also serve to substantially reduce chemical crosstalk between well environments when executing sequencing by synthesis reactions with arrays of wells comprising nucleic acid template beads. For example, FIG. 8 is an electron micrograph image of one embodiment of an array of reaction wells comprising buffering beads 805 and nucleic acid beads 810. It should be noted there is a relative size difference between beads 805 and 810 in FIG. 8, however the sizes and relative difference in size depicted in the image should not be considered as limiting. As illustrated in FIG. 8, nucleic acid bead 810 occupies most of the available space in a reaction well and buffering bead 805 can sit on top of nucleic acid bead 810 often in a corner area (i.e. in a square, rectangular, other shape with angular corners comprising sufficient dimension, circular, or oval shape comprising sufficient dimension) that allows a portion of buffering bead to settle beneath the plane of the well opening while on top of nucleic acid bead 810. Further, buffering bead 805 may also sit on top of the wall structure which defines the wells but is in fluid communication with the flow cell. FIG. 8 also illustrates one or more nucleic acid template beads 810 positionaly located outside of the wells which are capable of incorporating nucleic acid species and generating H⁺ signals that can enter one or more wells in the local area where buffering beads 805 can substantially reduce or eliminate the detection of the H⁺ by the ISFET sensors within those local wells. It will also be appreciated that in some embodiments the buffering species may be associated with well structure, such as for example associating the buffering species with the planar surface above the well openings or in the top most region of the internal wall surfaces of the well structures. Also in the same or alternative embodiments, buffering species could be included in wash solutions introduced into the reaction environment after each addition of a nucleotide species. In the described embodiments the bead substrates could be effectively replaced by the spatial arrangement or delivery strategy of the buffering species.

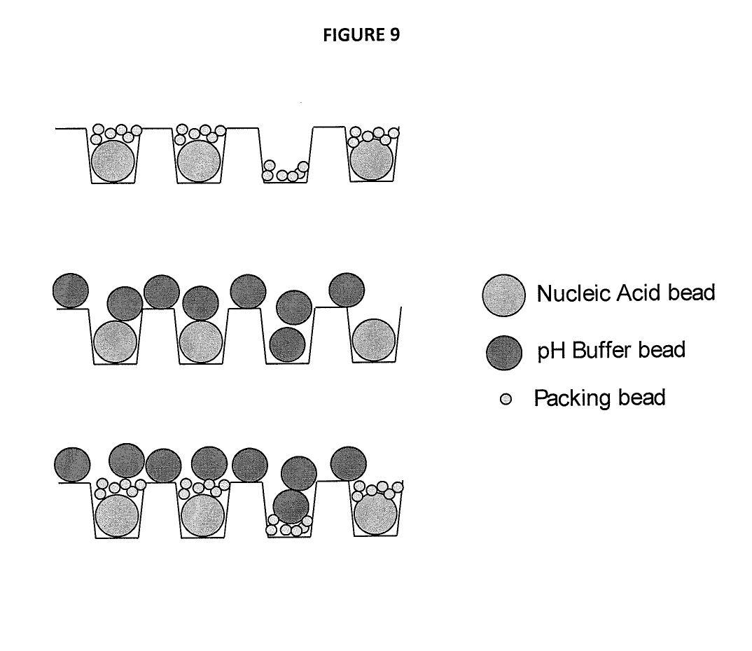

In the same or alternative embodiments, additional beads species may also be employed that may provide various functional advantages although a functional advantage is not necessarily required. One such species may be a bead species which has little or no pH buffering characteristics and may include a dimension that is smaller than other bead species employed, a dimension that is equivalent to the other bead species employed, a dimension that is greater than other bead species employed, or some combination thereof. For example, in some embodiments a bead species with substantially no pH buffering characteristics may generally be referred to as a “packing bead” species and used in combination with a nucleic acid bead species and a high pH buffering bead species. For instance, FIG. 9 provides an illustrative example of a comparison with one embodiment of a packing bead species in wells alone, packing beads used in combination with nucleic acid bead species in wells, and packing beads used in combination with nucleic acid bead species and pH buffering bead species in wells. In the exemplary embodiments of FIG. 9, a bead layering strategy is employed. In a first 2 layer embodiment, a first layer comprises nucleic acid beads are positionaly located in a first layer nearest the bottom surface of the wells with a dimension that is close to the width of the wells and does not permit smaller bead species past and a second layer comprising packing beads above. In a second 2 layer embodiment, the nucleic acid beads are positionaly located in the first layer as in the first 2 layer embodiment and the pH buffering bead species are positionaly located in the second layer above the nucleic acid bead species. Further, in a 3 layer embodiment a layer of packing beads is positionaly located in the second layer between the first nucleic acid bead layer and a third layer comprising the pH buffering bead species positionaly located at the top.

In the same or alternative embodiments, bead species may also be used that have enzyme species bound thereon. Enzyme species may provide desirable functional characteristics in some embodiments, such as for instance, apyrase or pyrophosphatase (also referred to as Ppi-ase) enzymes that may be used to degrade excess nucleotide species and/or reaction byproducts. In some circumstances the byproducts of the apyrase or Ppi-ase degradation may produce usable molecules or desirable conditions. For example, enzyme beads may be positionaly located in a middle and/or top layer depending on the functional result desired.

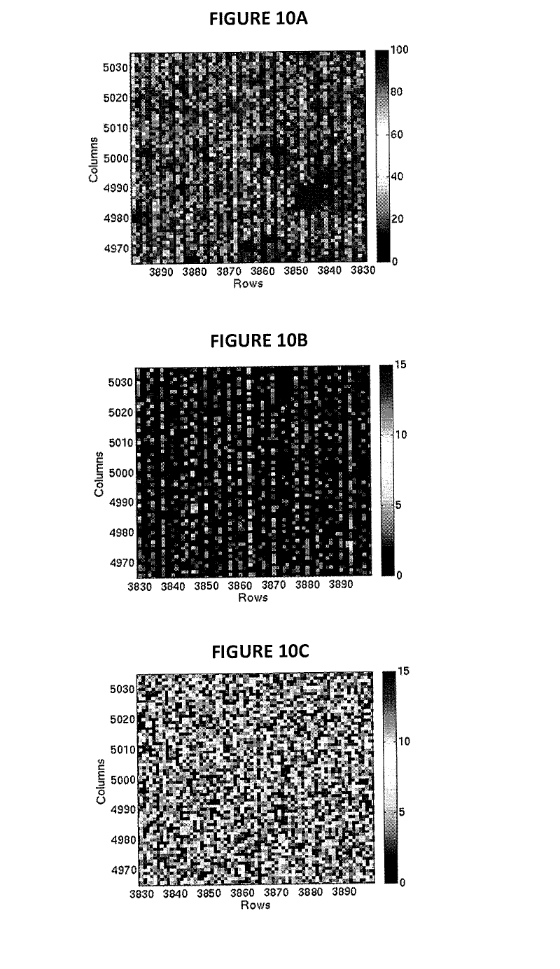

FIGS. 10A-C provide graphical examples of signals detected by an array of wells comprising ISFET sensors. More specifically, FIG. 10A demonstrates signals detected from the ISFET sensors in individual wells when using the first 2 layer embodiment of FIG. 9 with nucleic acid beads and packing beads. The signals are acquired immediately after a nucleotide incorporation event occurs releasing H⁺ ions where high levels of detected H⁺ ions detected by individual ISFET sensors are represented by Red pixels, and ISFET sensors which do not detect H⁺ ions (or relatively low levels of H⁺ ions) are represented by Blue pixels. It will be appreciated that there are almost no Blue pixels represented in FIG. 10A which means that there is a substantial amount of H⁺ ions migrating out of wells with the nucleic acid beads into neighboring wells that lack nucleic acid beads where ISFET sensor detects the migrating the H⁺ ions (i.e. chemical crosstalk).

FIG. 10B provides and graphical example of the second 2 layer embodiment described with respect to FIG. 9 with nucleic acid beads and pH buffering beads under the same condition of signal acquisition after nucleotide incorporation as FIG. 10A. Again, signals detected by an array of wells comprising ISFET sensors illustrate a result that is in contrast to the array signals of FIG. 10A. FIG. 10B shows a very high proportion of Blue pixels with almost no Red pixels indicating a high degree of buffering of the H⁺ ions migrating out of the wells and almost no chemical cross talk. However, in the example of FIG. 10B the H⁺ ions in the wells with nucleic acid beads are also substantially buffered resulting in very low, or no detectable H⁺ ions remaining (sometimes referred to a signal “quenching”). It will be appreciated that the amount of buffering by a pH buffering bead species may vary by amount or type of functional groups associated or other characteristic and that a bead species with a lower pH buffering characteristic could be employed preserving detectable signals in the wells comprising nucleic acid beads, and thus the example of FIG. 10B should not be considered as limiting.

Lastly, FIG. 10C provides a graphical example of the 3 layer embodiment described with respect to FIG. 9 again under the same condition of signal acquisition after nucleotide incorporation as FIGS. 10A and 10B. Those of ordinary skill will appreciate that the proportion of Red and Blue indicate that the combination of packing beads in a second layer and pH buffering beads in a top third layer act to minimize both chemical crosstalk between wells as well as quenching within wells with nucleic acid beads.

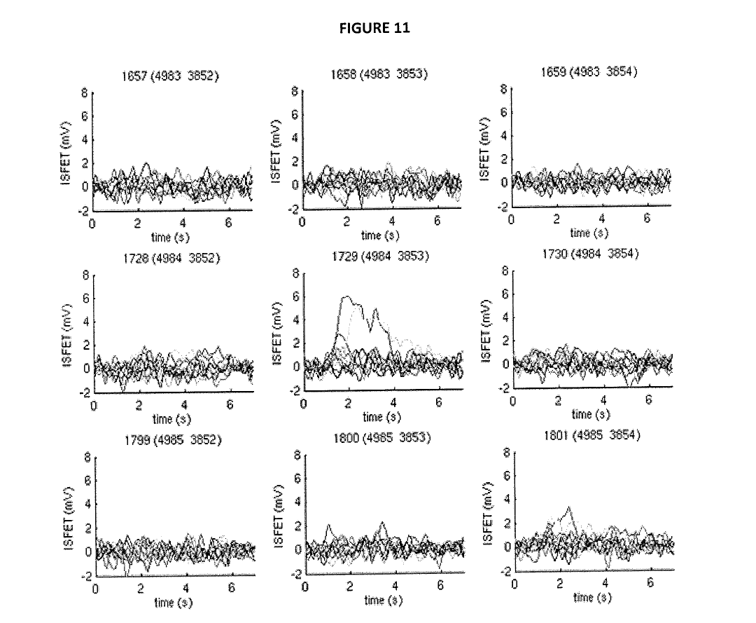

FIG. 11 provides another example of signals generated in a well from a nucleic acid template bead in the presence of pH buffering beads where neighboring wells show minimal spread of the signal from the nucleic acid template well. More specifically, FIG. 11 shows signals detected by ISFET sensors (in mV) over a signal acquisition time period from 9 wells with the centrally located well (well number 1729 in row 4984 and column 3853) comprising a nucleic acid template species. It is notable that the peaks of detected values in well 1729 occur within 2-4 seconds of introduction of the nucleotide species, and that wells surrounding well 1729 show almost no signal above background with the exception of some small signal in wells 1800 and 1801.

Additional benefits derived from the use of the high pH buffering beads include a greatly reduced time scale required for signal acquisition. For example, without pH buffering beads in the system the duration of a detectable signal in a well after incorporation of a nucleotide species can be on the order of about 20 seconds, whereas in the presence of high pH buffering beads the duration of detectable signal can be reduced to a range of about 2-4 seconds (as discussed above with respect to the illustrative example of FIG. 11). In the present example, the pH buffering characteristics of the beads reduces the amount of H⁺ ions available for detection by the ISFET sensor as a function of time so that signals can be acquired and processed very quickly. Also in the present example, the rise time of the signal waveform is generally not affected to a substantial degree.

Another additional benefit provided by use of the high pH buffering bead embodiments in semiconductor based sequencing systems includes a buffering effect on the background signal that may be created by the flow of a nucleotide species or other flow of reagent that contributes to a different pH during a signal acquisition period for nucleotide species incorporation. For example, a flow of a wash solution may comprise pH 8.0 and a flow of a nucleotide species may comprise pH 8.1 and subsequently a flow of wash solution at pH 8.0, where the difference in pH in the flow of nucleotide species and wash solution creates an undesirable background signal from the pH difference of the flows occurring at the same time as the signal from the incorporation of the nucleotide species is being acquired. The use of pH buffering beads greatly reduces the background signal from the pH differences by buffering the pH change associated with the different flows from pH 8.0 to 8.1. The result is an improvement in the ability to discriminate the signal from the nucleotide species incorporation from the background signal.

Having described various embodiments and implementations, it should be apparent to those skilled in the relevant art that the foregoing is illustrative only and not limiting, having been presented by way of example only. Many other schemes for distributing functions among the various functional elements of the illustrated embodiments are possible. The functions of any element may be carried out in various ways in alternative embodiments.

What is claimed is:

1. A method for sequencing a species of nucleic acid template using pH inert reference sensors, comprising the steps of:
(a) introducing a nucleotide species to an array of wells wherein a plurality of the wells comprise a species of nucleic acid template and a plurality of the wells comprise a plurality of functional groups with a high pH buffering characteristic, and wherein in at least a first well a polymerase species incorporates the nucleotide species into a plurality of strands complementary to the species of nucleic acid template disposed in the first well and results in a release of a plurality of hydrogen ions;
(b) detecting a signal in the first well, wherein the signal is responsive to the hydrogen ions and one or more noise sources;
(c) detecting a signal in a second well comprising the functional groups with the high pH buffering characteristic, wherein the signal is responsive to the one or more noise sources; and
(d) subtracting the second well signal from the first well signal to generate a corrected signal associated with the detected hydrogen ions.

2. The method of claim 1, wherein:
the species of nucleic acid template are disposed on beads.

3. The method of claim 1, wherein:
the functional groups comprising the high pH buffering characteristic are disposed on beads.

4. The method of claim 1, wherein:
the functional groups comprise carboxylic acid functional groups.

5. The method of claim 1, wherein:
the functional groups comprising the high pH buffering characteristic are coated on a sensor element in one or more of the wells.

6. The method of claim 5, wherein:
the sensor element comprises an ISFET sensor.

7. The method of claim 1, wherein:
the nucleotide species are introduced in an aqueous solution

8. The method of claim 1, wherein:
the array of wells are in fluid communication with each other.

9. The method of claim 8, wherein:
the fluid communication is provided by a flow cell environment.

10. The method of claim 9, wherein:
the nucleotide species is introduced into the flow cell environment.

11. The method of claim 1, wherein:
the signal responsive to the hydrogen ions and one or more noise sources and the signal responsive to one or more noise sources are detected by ISFET sensors.

12. The method of claim 1, wherein:
the noise sources comprise temperature and electrical signals

13. The method of claim 1, wherein:
the first well signal and the second well signal comprise a plurality of detected mV signals over time.

14. The method of claim 1, further comprising:
(e) determining a base call based, at least in part, upon the corrected signal.

15. The method of claim 1, further comprising:
repeating steps (a)-(e) for a plurality of sequence positions of the species of nucleic acid template.

16. A system for sequencing a species of nucleic acid template using pH inert reference sensors, comprising the steps of:
(a) a flow cell that provides fluid communication to an array of wells wherein the flow cell operatively couples to a fluidic subsystem that introduces a nucleotide species to a plurality of the wells that comprise a species of nucleic acid template and a plurality of the wells that comprise a plurality of functional groups with a high pH buffering characteristic, and wherein in at least a first well a polymerase species incorporates the nucleotide species into a plurality of strands complementary to the species of nucleic acid template disposed in the first well and results in a release of a plurality of hydrogen ions;
(b) an ISFET sensor in the first well that detects a signal responsive to the hydrogen ions and one or more noise sources;
(c) an ISFET sensor in a second well comprising the functional groups with the high pH buffering characteristic that detects a signal responsive to the one or more noise sources; and
(d) a computer comprising executable code stored thereon that subtracts the second well signal from the first well signal to generate a corrected signal associated with the detected hydrogen ions.

17. The system of claim 16, wherein:
the species of nucleic acid template are disposed on beads.

18. The system of claim 16, wherein:
the functional groups comprising the high pH buffering characteristic are disposed on beads.

19. The system of claim 16, wherein:
the functional groups comprise carboxylic acid functional groups.

20. The system of claim 16, wherein:
the functional groups comprising the high pH buffering characteristic are coated on a sensor element in one or more of the wells.

21. The system of claim 16, wherein:
the nucleotide species are introduced in an aqueous solution

22. The system of claim 16, wherein:
the noise sources comprise temperature and electrical signals

23. The system of claim 16, wherein:
the first well signal and the second well signal comprise a plurality of detected mV signals over time.

24. The system of claim 16, wherein:
the computer and executable code determines a base call based, at least in part, upon the corrected signal.

25. A method for sequencing a species of nucleic acid template using pH inert reference sensors, comprising the steps of:
(a) distributing a plurality of beads comprising a species of nucleic acid template disposed thereon and a plurality of beads comprising a high pH buffering characteristic into individual wells of an array of wells in a flow cell environment;
(b) introducing into the flow cell environment a nucleotide species complementary to the species of nucleic acid template disposed on the bead in at least a first well, wherein a polymerase species incorporates the nucleotide species into a plurality of complementary strands that results in a release of a plurality of hydrogen ions;
(c) detecting a signal in the first well, wherein the signal is responsive to the hydrogen ions and one or more noise sources;
(d) detecting a signal in a second well comprising one or more of the high pH buffering beads, wherein the signal is responsive to the one or more noise sources; and
(e) subtracting the second well signal from the first well signal to generate a corrected signal associated with the detected hydrogen ions.

26. The method of claim 25, wherein:
the high pH buffering characteristic is enabled by carboxylic acid functional groups attached to the surface areas of the beads.

27. The method of claim 26, wherein:
the functional groups comprising a high pH buffering characteristic reduce chemical cross talk between individual wells of the array of wells.

28. The method of claim 25, wherein:
the step of distributing further comprises distributing a plurality of packing beads into the individual wells in a layer above the nucleic acid bead and below the beads comprising a high pH buffering characteristic.

29. An array of ISFET sensors, comprising:
one or more detection well structures each associated with at least one ISFET detector positioned at a bottom region of each of the first well structures and are sensitive to change of pH in a fluid; and
one or more of reference well structures with at least one ISFET detector positioned at a bottom region of each of the reference well structures and in fluid communication with the detection well structures, wherein the reference well structures comprise a high buffering bead disposed within, and wherein the ISFET detectors in the reference well structures are insensitive to change of pH in the fluid.

30. An array of ISFET sensors, comprising:
one or more first ISFET detectors sensitive to change of pH in a fluid; and
one or more of reference ISFET detectors in fluid communication with the first ISFET detectors, wherein the reference ISFET detectors comprising a coating of a pH buffering functional group, and wherein the reference ISFET detectors are insensitive to a change of pH in the fluid.
